(19) United States
(12) Patent Application Publication
(10) Pub. No.: US 2025/0344035 A1
(43) Pub. Date: Nov. 6, 2025

(54) SYSTEMS AND METHODS FOR REAL-TIME USER POSITIONING

(71) Applicant: PLUME DESIGN, INC., Palo Alto, CA (US)

(72) Inventors: Fahri DINER, Zurich (CH); Miroslav SAMARDZIJA, Mountain View, CA (US); Liem Hieu Dinh VO, San Jose, CA (US); Kiran EDARA, Cupertino, CA (US); Kai HU, Union City, CA (US); Todd ROYCE, Redwood City, CA (US)

(21) Appl. No.: 18/652,925

(22) Filed: May 2, 2024

(51) Int. Cl. H04W 4/021; A61B 5/00; H04W 4/06; H04W 4/90

(52) U.S. Cl. CPC H04W 4/021; A61B 5/6826; H04W 4/06; H04W 4/90

(57) ABSTRACT

Disclosed are systems and methods that provide a decision-intelligence (DI)-based, computerized framework for advanced device and/or user localization upon the occurrence of an event. The disclosed framework can detect the occurrence of an emergency, which can be localized and/or personal to a user or a global/regional event, and detect the presence and precise location of a user. The framework can enable the scanning of a geographical area for the presence of devices, which can be a specific type (e.g., smart ring). Upon detecting the presence of a ring, for example, the signal information from the emergency broadcast signal from the device can be analyzed and leveraged to pinpoint a location from the scanning device to the ring. This, therefore, enables a first responder to pinpoint the position at a location so they can be timely rescued. Accordingly, the disclosed framework improves how devices can operate for a live-saving purpose.

### FIELD OF THE DISCLOSURE

The present disclosure is generally related to location management and pin-pointing of users and/or the users' associated devices, and more particularly, to a decision intelligence (DI)-based computerized framework for deterministically performing advanced device localization during and/or upon the occurrence of an event.

### SUMMARY OF THE DISCLOSURE

By way of background, during emergencies, such as earthquakes, for example, locating individuals can pose significant challenges, particularly when network connectivity is disrupted. In scenarios where the network connected to their devices is down, traditional methods like GPS tracking and/or mobile network triangulation become ineffective. This presents a technical deficiency in the current emergency response systems, as reliance on digital communication and location-based services becomes futile. Manual search efforts become the primary method of locating individuals, relying on first responders and community volunteers to physically search affected areas (which can include using listing tools to find void spaces and/or trained dogs that can navigate an area to sniff for survivors, for example). However, this approach is time-consuming, resource-intensive and may not always guarantee successful outcomes, especially in large-scale disasters or remote locations.

For example, between 1998-2017, earthquakes caused nearly 750 000 deaths globally, more than half of all deaths related to natural disasters. More than 125 million people were affected by earthquakes during this time period, meaning they were injured, made homeless, displaced or evacuated during the emergency phase of the disaster.

Thus, the current lack of alternative technological solutions for locating individuals when network connectivity is lost underscores the need for innovation and robust contingency plans in emergency response systems.

According to some embodiments, the disclosed systems and methods provide a novel framework for leveraging modern technology to detect the occurrence of an emergency, which can be localized and/or personal to a user or a global/regional event, and detect the presence and precise location of a user. As discussed herein, in some embodiments, the disclosed framework can enable the scanning of a geographical area for the presence of devices, which can be a specific type (e.g., smart ring, for example). Upon detecting the presence of a ring, for example, the signal information from the emergency broadcast signal from the device can be analyzed and leveraged to pinpoint a location (e.g., direction and distance) from the scanning device to the ring. This, therefore, enables a first responder to pinpoint the position of a user (at x, y, z coordinates) at a location so they can be timely rescued. Accordingly, the disclosed technology improves how devices can operate for a live-saving purpose.

According to some embodiments, a method is disclosed for a DI-based computerized framework for DSPs to deterministically perform advanced device localization during and/or upon the occurrence of an event. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for deterministically performing advanced device localization during and/or upon the occurrence of an event.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary workflow according to some embodiments of the present disclosure;

FIG. 4 depicts an exemplary implementation of an architecture according to some embodiments of the present disclosure;

FIG. 5 depicts an exemplary implementation of an architecture according to some embodiments of the present disclosure; and

FIG. 6 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ different architectures or may be compliant or compatible with different protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

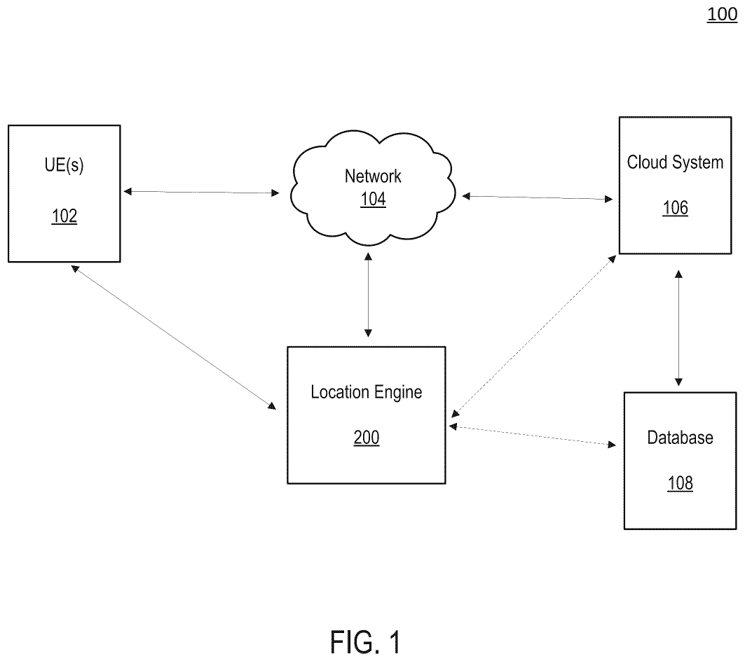

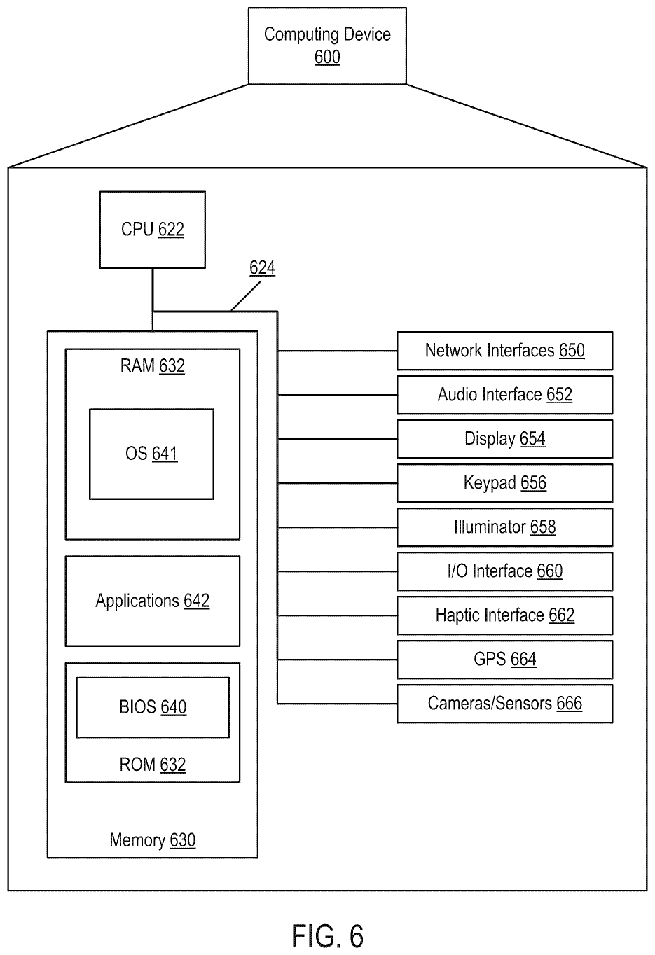

Certain embodiments and principles will be discussed in more detail with reference to the figures. With reference to FIG. 1, system 100 is depicted which includes user equipment (UE) 102 (e.g., a client device, as mentioned above and discussed below in relation to FIG. 6), network 104, cloud system 106, database 108 and location engine 200. It should be understood that while system 100 is depicted as including such components, it should not be construed as limiting, as one of ordinary skill in the art would readily understand that varying numbers of UEs, access point (AP) devices, peripheral devices, sensors, cloud systems, databases and networks can be utilized; however, for purposes of explanation, system 100 is discussed in relation to the example depiction in FIG. 1.

According to some embodiments, UE 102 can be any type of device, such as, but not limited to, a mobile phone, tablet, laptop, sensor, IoT device, wearable device, autonomous machine, smart television, media streaming device, game console, and any other device equipped with a cellular or wireless or wired transceiver.

In some embodiments, peripheral devices (not shown) can be connected to UE 102, and can be any type of peripheral device, such as, but not limited to, a wearable device (e.g., smart ring, smart watch, for example), printer, speaker, sensor, and the like. In some embodiments, a peripheral device can be any type of device that is connectable to UE 102 via any type of known or to be known pairing mechanism, including, but not limited to, WiFi, Bluetooth™, Bluetooth Low Energy (BLE), NFC, and the like.

According to some embodiments, UE 102 can correspond to an AP device, which is a device that creates and/or provides a wireless local area network (WLAN) for a location, for which a UE can connect thereto. According to some embodiments, the AP device can be, but is not limited to, a router, switch, hub, gateway, extender and/or any other type of network hardware that can project a WiFi signal to a designated area.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service provider and/or network provider from where services and/or applications may be accessed, sourced or executed from. For example, system 106 can represent the cloud-based architecture associated with a smart home or network provider (e.g., Plume Design®), which has associated network resources hosted on the internet or private network (e.g., network 104), which enables (via engine 200) the network management discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, and the services and applications provided by cloud system 106 and/or location engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

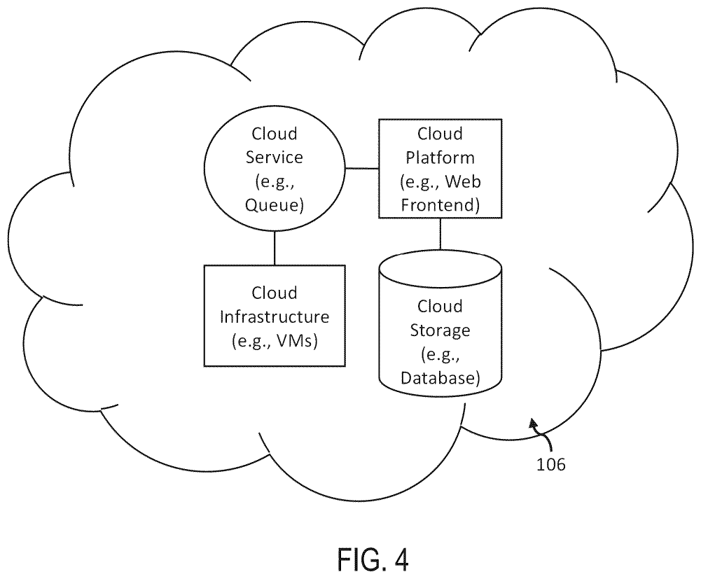

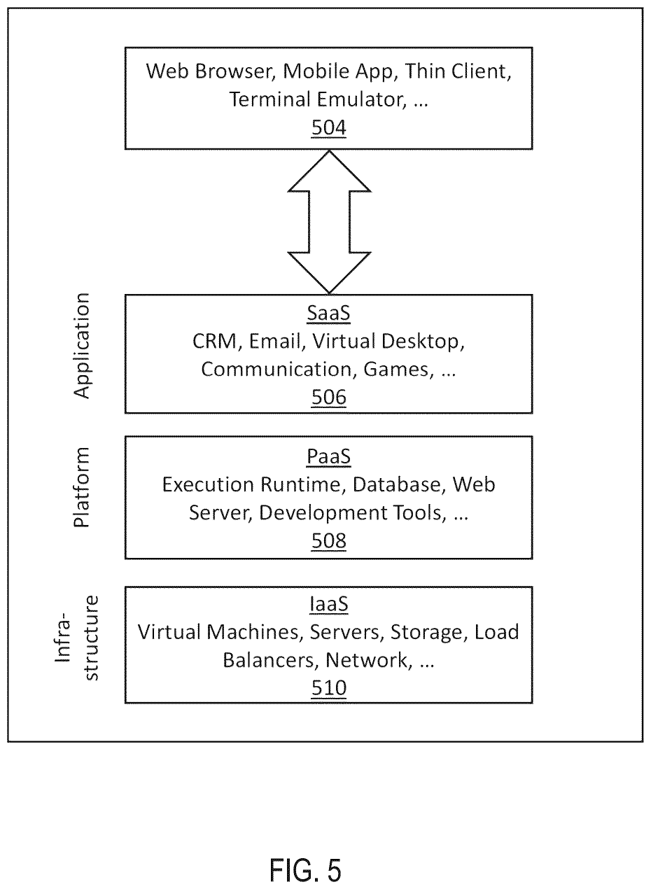

Turning to FIGS. 4 and 5, in some embodiments, the exemplary computer-based systems/platforms, the exemplary computer-based devices, and/or the exemplary computer-based components of the present disclosure may be specifically configured to operate in a cloud computing/architecture 106 such as, but not limiting to: infrastructure as a service (IaaS) 510, platform as a service (PaaS) 508, and/or software as a service (SaaS) 506 using a web browser, mobile app, thin client, terminal emulator or other endpoint 504. FIGS. 4 and 5 illustrate schematics of non-limiting implementations of the cloud computing/architecture(s) in which the exemplary computer-based systems for administrative customizations and control of network-hosted application program interfaces (APIs) of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, structured query language (SQL). According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository.

Location engine 200, as discussed above and further below in more detail, can include components for the disclosed functionality. According to some embodiments, location engine 200 may be a special purpose machine or processor, and can be hosted by a device on network 104, within cloud system 106 and/or on UE 102. In some embodiments, engine 200 may be hosted by a server and/or set of servers associated with cloud system 106.

According to some embodiments, as discussed in more detail below, location engine 200 may be configured to implement and/or control a plurality of services and/or microservices, where each of the plurality of services/microservices are configured to execute a plurality of workflows associated with performing the disclosed network management. Non-limiting embodiments of such workflows are discussed and provided below.

According to some embodiments, as discussed above, location engine 200 may function as an application provided by cloud system 106. In some embodiments, engine 200 may function as an application installed on a server(s), network location and/or other type of network resource associated with system 106. In some embodiments, engine 200 may function as an application installed and/or executing on UE 102. In some embodiments, such application may be a web-based application accessed by UE 102, and/or other devices (e.g., peripheral devices, for example) accessible over network 104 from cloud system 106. In some embodiments, engine 200 may be configured and/or installed as an augmenting script, program or application (e.g., a plug-in or extension) to another application or program provided by cloud system 106 and/or executing on UE 102.

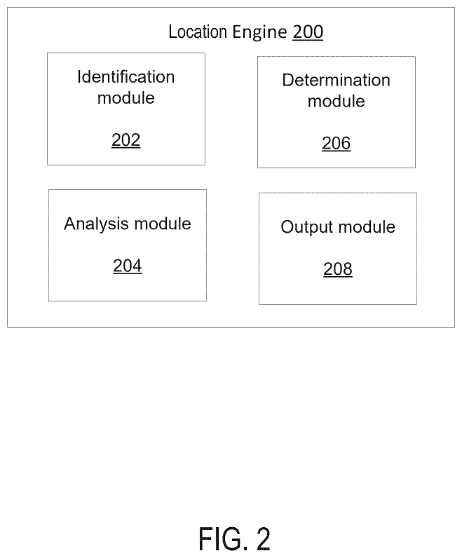

As illustrated in FIG. 2, according to some embodiments, location engine 200 includes identification module 202, analysis module 204, determination module 206 and output module 206. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

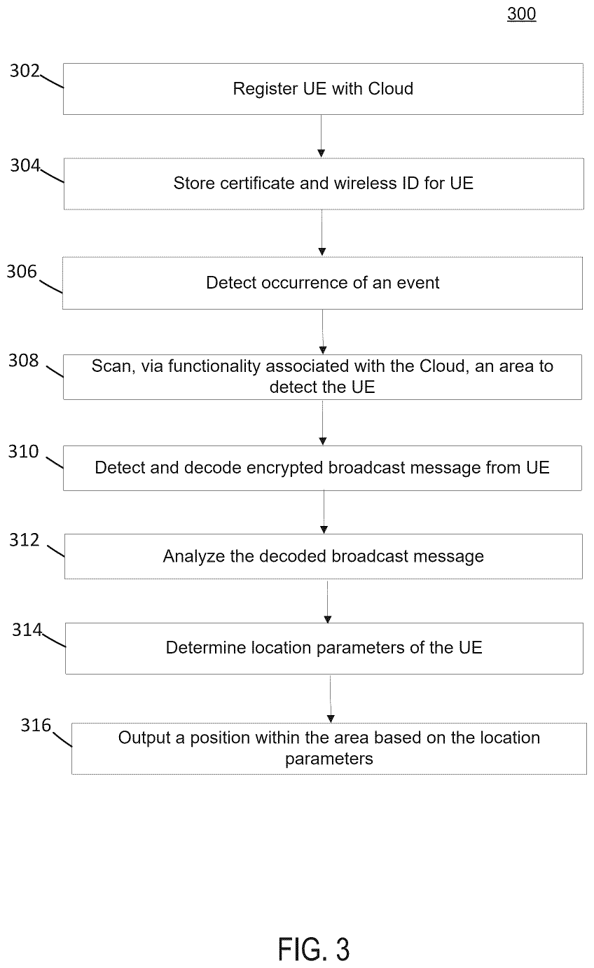

Turning to FIG. 3, Process 300 provides non-limiting example embodiments for the disclosed localization functionality for users and their devices during and/or at a time proximate to an event (e.g., an emergency, for example, an earthquake). As provided below, the disclosed framework's configuration and implementation can provide a computerized suite of location tools for locating the geographical positioning of a user and/or their device(s).

According to some embodiments, Steps 302-310 of Process 300 can be performed by identification module 202 of location engine 200; Step 312 can be performed by analysis module 204; Step 314 can be performed by determination module 206; and Step 316 can be performed by output module 206.

According to some embodiments, Process 300 begins with Step 302 where engine 200 can enable a UE to register with the Cloud. According to some embodiments, to register a UE, such as a smart ring, as discussed herein, with an account on the Cloud, a process through a companion mobile application and/or a web interface can be initiated. For example, in some embodiments, upon launching the application and/or accessing the website, an account can be created by providing personal information, such as, but not limited to, name, email address and password, location, and the like. Once the account is set up, the device registration section within the application and/or website can be initiated, which enables prompts and inputs to add the smart ring to the account.

According to some embodiments, this can involve activating the UE by pressing a designated button and/or scanning a QR code displayed on the smart ring's packaging and/or within its companion app. The application then establishes a connection between the smart ring and the user's account on the Cloud server, associating unique identifiers and authentication tokens to ensure secure communication. Once registered, the smart ring's settings, data and functionalities can be remotely accessed and managed via the account on the Cloud.

According to some embodiments, such registration can involve monitoring and/or collecting data related to the user. That is, for example, in some embodiments, the UE/smart ring can collect a variety of vital signs and health metrics from a user, providing valuable insights into their well-being. Some of the vitals measured can include, but are not limited to, heart rate, heart rate variability (HRV), blood oxygen levels, body temperature and the like.

For example, heart rate monitoring can be achieved through optical sensors embedded within the ring, which emit light onto the skin and measure the variations in light absorption caused by blood flow.

According to some embodiments, such vitals can be continuously collected by the smart ring throughout the day and night, and the data can be synchronized with the account on in the Cloud.

In Step 304, engine 200 can store a certificate and wireless identifier (ID) for the UE/smart ring. According to some embodiments, a smart ring stored in the Cloud can utilize various types of certificates and wireless IDs for authentication and security purposes. For example, the certificate can be, but is not limited to, an secure sockets layer (SSL)/transport layer security (TLS) certificate, which can be used to establish a secure connection between the smart ring and a Cloud server, ensuring that data transmitted between them is encrypted and protected from unauthorized access.

In some embodiments, the smart ring may have a unique wireless ID, such as a MAC address or an RFID tag, which can be registered with the cloud account to uniquely identify the device and facilitate secure communication. These certificates and wireless IDs play a crucial role in verifying the identity of the smart ring and ensuring that only authorized users can access its functionalities and data stored in the cloud. Moreover, as provided below, such information can be leveraged to identify the UE upon the occurrence of an emergency event.

In Steps 306-316, Process 300 provides the functionality for which an registered UE can be utilized to locate its wearer (e.g., the corresponding user). As mentioned above, such location may be based on a personalized event (e.g., the user is presumed missing, and this can be used to locate them) and/or a global/regional event (e.g., an earthquake, for example). Moreover, while it will be discussed in relation to locating a single user, the disclosed systems and methods discussed herein should not be construed as limiting, as one of skill in the art would recognize that the disclosed functionality can be expanded to identify and location multiple UEs, which can be performed simultaneously for a set of UEs.

Turning to Step 306, the occurrence of such event(s) can be performed by engine 200. Such detection can be based on, but not limited to, a notification, social media activity, a request, an instruction, and the like. For example, engine 200 can detect a post on social media that indicates an earthquake was detected in Sacramento, CA. Thus, in some embodiments, engine 200 can function to scan network activity data to determine whether an event is detected as occurring, then localize the approach for which the local users can be accounted for and/or located. In some embodiments, upon occurrence of the emergency, the network may be rendered inaccessible by the UE and/or other devices on the network.

In some embodiments, engine 200 can detect an emergency situation (e.g., an earthquake) through various connected to and/or affiliated sensors (e.g., on and/or connected to UE 102, for example) and algorithms designed to recognize specific patterns or anomalies associated with such events. For example, for earthquakes specifically, accelerometers on the UE and/or peripheral UEs can be used to detect sudden changes in motion or vibration that are characteristic of seismic activity. These accelerometers measure changes in acceleration caused by ground motion, and when the device detects significant shaking or vibration beyond a certain threshold, it can trigger an alert indicating a potential earthquake.

In addition to accelerometers, UEs may also incorporate other sensors such as gyroscopes, magnetometers and/or barometers to provide supplementary data for detecting earthquakes or assessing their severity. For example, gyroscopes can detect rotational movements, while magnetometers can detect changes in magnetic fields that may occur during seismic activity. Barometers can also detect changes in air pressure, which may indicate the passage of a pressure wave associated with an earthquake.

Furthermore, advanced artificial intelligence/machine learning (AI/ML) algorithms can be employed to analyze sensor data in real-time and distinguish between normal environmental vibrations and those indicative of an earthquake. By continuously monitoring sensor data and analyzing it for specific seismic signatures, devices can accurately detect and respond to emergency situations such as earthquakes, providing timely alerts to users and authorities to take appropriate action and ensure safety.

In Step 308, engine 200 can enable the scanning of an area to detect the UE. For example, a first responder can execute an application program interface (API) call to an application and/or interface associated with the Cloud, whereby the device of the first responder can enable scanning for broadcasted signals from UEs in the region. Such broadcast signals can include, but are not limited to, account data for the UE, collected and/or current vitals from the UE, location information (as derived from signals emitted from the UE), and the like, as discussed infra.

And, in Step 310, based on the scanning, the broadcast message from the UE/smart ring, which can be encrypted, as discussed below, can be detected and decoded.

In some embodiments, to enable a device, such as a smartphone, to scan a location for broadcast messages from a smart ring, the smart ring and the first responder device can leverage compatible communication capabilities and protocols, such as, for example, Bluetooth Low Energy (BLE) technology.

According to some embodiments, such BLE broadcast messages can be periodically communicated, and they can include information about the wearer's health status, location, and/or other customized alerts. When the first responder device comes into proximity with the smart ring (e.g., within a distance where a BLE signal can be detected at a threshold level), its BLE receiver detects the broadcast messages being transmitted by the smart ring. The first responder's device can execute the API/application to process the smart ring message(s), whereby operations for extracting relevant information and presenting it to the user through a user interface (UI) and/or triggering predefined actions based on the content of the message can be performed (e.g., Steps 310-316, discussed infra).

Thus, for example, via Step 310, the first responder's scanning actions can involve searching a geographical range of surroundings for broadcast messages from a smart ring, enabling various applications such as health monitoring, location tracking, and emergency alerts.

According to some embodiments, during an emergency, beyond BLE-type broadcast messages, more sophisticated communication protocols may be employed to disseminate critical information efficiently and securely. In some embodiments, encrypted broadcast messages, which are designed to transmit sensitive information to designated recipients while protecting the data from unauthorized access or interception, can be utilized.

In the context of the above smart ring-first responder scenario, encrypted broadcast messages can be used to transmit emergency alerts, location coordinates, or health status updates to nearby devices or designated emergency responders. These messages can be encrypted using strong encryption algorithms and keys shared only among authorized parties, ensuring that the information remains confidential and secure throughout transmission. For example, such algorithms/keys can include, but are not limited to, Advanced Encryption Standard (AES), Rivest Cipher (RC), Triple Data Encryption Standard (3DES), Elliptic Curve Cryptography (ECC), Secure Hash Algorithms (SHA), and the like, or some combination thereof.

In some embodiments, to receive and decrypt these encrypted broadcast messages, devices such as smartphones or other wearable devices would need to have the appropriate decryption keys and software capabilities. In some embodiments, such capabilities can be provided via the application/API executing and/or accessed via the first responder's phone.

Accordingly, in some embodiments, upon receiving an encrypted broadcast message, the first responder device (e.g., recipient device) can use its decryption key to decrypt the message and access the underlying information. Such approach offers several advantages during emergencies. Firstly, encrypted broadcast messages provide a secure means of communication, protecting sensitive information from being intercepted or tampered with by malicious actors. Secondly, by broadcasting messages to nearby devices, encrypted communication enables rapid dissemination of critical information to affected individuals or emergency responders, facilitating prompt response and assistance.

Accordingly, encrypted broadcast messages offer a robust and secure method of communication during emergencies, ensuring that vital information can be transmitted quickly and securely to those who need it most.

In some embodiments, upon the message being received by the Cloud connected first responder device, it can be stored in database 108, as discussed above.

In Step 312, the message can then be analyzed, which can reveal vitals about the wearer (e.g. user) and/or location parameters, which can include signal strength and the like. In some embodiments, such parameters can further include, but are not limited to, GPS coordinates, BLE beacon IDs, cell tower information, barometric pressure (e.g., provide altitude information), time stamps, and the like.

According to some embodiments, Step 312's analysis can involve engine 200 executing a specific trained AI/ML model, a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 200 may be configured to utilize one or more AI/ML techniques selected from, but not limited to, computer vision, feature vector analysis, decision trees, boosting, support-vector machines, neural networks, nearest neighbor algorithms, Naive Bayes, bagging, random forests, logistic regression, and the like.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
e. apply the trained model to process the newly received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

Thus, based on the analysis of the WiFi data, in Step 314, engine 200 can determine the vitals and/or location parameters of the user. Such vitals, for example, can indicate whether the user is alive and/or their health status. This can aid in prioritizing users with regard to which users need rescuing more than others, for example. Moreover, in some embodiments, the location parameter determination can provide an indication as to the direction and distance of the UE/smart ring (e.g., user/wearer) from the first responder device, which can enable the first responder to pinpoint their location to execute the search. For example, if a user is buried in rubble, the location parameters can indicate that the user is 10 feet away (in the positive x direction, and 5 feet down, which can be respective to the first responder's position and/or sea level, for example). In some embodiments, such information can indicate the vitals of the user (e.g., HRV is below a threshold and person requires immediate attention, for example).

Accordingly, in Step 316, engine 200 can be compiled into a renderable message, which can be output via, but not limited to, a user interface (UI), audio message, video message, multi-media message, and the like. For example, a graphical display can display the direction and distance on a UI of the first responder's device. In another example, an audible message can be output relaying the information to the first responder. And, in another non-limiting example, a SMS message can be generated and communicated, which indicates the vitals, user/UE ID and/or location parameters, which can be displayed as text or as a hyperlinked message that causes such information to be displayed on the UI within the first responder's display.

FIG. 6 is a schematic diagram illustrating a client device showing an example embodiment of a client device that may be used within the present disclosure. Client device 600 may include many more or less components than those shown in FIG. 6. However, the components shown are sufficient to disclose an illustrative embodiment for implementing the present disclosure. Client device 600 may represent, for example, UE 102 discussed above at least in relation to FIG. 1.

As shown in the figure, in some embodiments, Client device 600 includes a processing unit (CPU) 622 in communication with a mass memory 630 via a bus 624. Client device 600 also includes a power supply 626, one or more network interfaces 650, an audio interface 652, a display 654, a keypad 656, an illuminator 658, an input/output interface 660, a haptic interface 662, an optional global positioning systems (GPS) receiver 664 and a camera(s) or other optical, thermal or electromagnetic sensors 666. Device 600 can include one camera/sensor 666, or a plurality of cameras/sensors 666, as understood by those of skill in the art. Power supply 626 provides power to Client device 600.

Client device 600 may optionally communicate with a base station (not shown), or directly with another computing device. In some embodiments, network interface 650 is sometimes known as a transceiver, transceiving device, or network interface card (NIC).

Audio interface 652 is arranged to produce and receive audio signals such as the sound of a human voice in some embodiments. Display 654 may be a liquid crystal display (LCD), gas plasma, light emitting diode (LED), or any other type of display used with a computing device. Display 654 may also include a touch sensitive screen arranged to receive input from an object such as a stylus or a digit from a human hand.

Keypad 656 may include any input device arranged to receive input from a user. Illuminator 658 may provide a status indication and/or provide light.

Client device 600 also includes input/output interface 660 for communicating with external. Input/output interface 660 can utilize one or more communication technologies, such as USB, infrared, Bluetooth™, or the like in some embodiments. Haptic interface 662 is arranged to provide tactile feedback to a user of the client device.

Optional GPS transceiver 664 can determine the physical coordinates of Client device 600 on the surface of the Earth, which typically outputs a location as latitude and longitude values. GPS transceiver 664 can also employ other geo-positioning mechanisms, including, but not limited to, triangulation, assisted GPS (AGPS), E-OTD, CI, SAI, ETA, BSS or the like, to further determine the physical location of client device 600 on the surface of the Earth. In one embodiment, however, Client device 600 may through other components, provide other information that may be employed to determine a physical location of the device, including for example, a MAC address, Internet Protocol (IP) address, or the like.

Mass memory 630 includes a RAM 632, a ROM 634, and other storage means. Mass memory 630 illustrates another example of computer storage media for storage of information such as computer readable instructions, data structures, program modules or other data. Mass memory 630 stores a basic input/output system (“BIOS”) 640 for controlling low-level operation of Client device 600. The mass memory also stores an operating system 641 for controlling the operation of Client device 600.

Memory 630 further includes one or more data stores, which can be utilized by Client device 600 to store, among other things, applications 642 and/or other information or data. For example, data stores may be employed to store information that describes various capabilities of Client device 600. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 600.

Applications 642 may include computer executable instructions which, when executed by Client device 600, transmit, receive, and/or otherwise process audio, video, images, and enable telecommunication with a server and/or another user of another client device. Applications 642 may further include a client that is configured to send, to receive, and/or to otherwise process gaming, goods/services and/or other forms of data, messages and content hosted and provided by the platform associated with engine 200 and its affiliates.

According to some embodiments, certain aspects of the instant disclosure can be embodied via functionality discussed herein, as disclosed supra. According to some embodiments, some non-limiting aspects can include, but are not limited to the below method aspects, which can additionally be embodied as system, apparatus and/or device functionality:

Aspect 1. A method comprising:
scanning, by a device, an area associated with a geographical location, the geographical location being associated with an event, the scanning comprising monitoring for detected signals from user equipment (UE) associated with a user;
detecting, by the device based on the scanning, a broadcast message from the UE;
analyzing, by the device, the broadcast message;
determining, by the device, based on the analysis, information related to vitals of the user and a location of the user within the area; and
outputting, by the device, the determined information, the output information enabling retrieval of the user to safety in response to the event.

Aspect 2. The method of claim 1, wherein the broadcast message is an encrypted broadcast message communicated by the UE.

Aspect 3. The method of claim 2, further comprising:
decoding the encrypted broadcast message upon detection of the encrypted broadcast message, wherein the analysis is performed in accordance with the decoded broadcast message.

Aspect 4. The method of claim 1, further comprising:
detecting, over a network, the event, the detection corresponding to a time proximate to the event, wherein the scanning of the area is based on the detection of the event.

Aspect 5. The method of claim 1, wherein the event is an emergency, wherein, upon occurrence of the emergency, the network is rendered inaccessible by the UE.

Aspect 6. The method of claim 1, wherein the analysis of the broadcast message is based on a signal strength of the broadcast message, wherein the location of the user within the area is determined at least on the analysis of the signal strength, wherein the location of the user corresponds to a distance and direction from the device.

Aspect 7. The method of claim 1, wherein the device associated with a first responder, wherein the device executes an application associated with a Cloud, wherein the user of the UE has an account with the Cloud.

Aspect 8. The method of claim 1, wherein the UE is a smart ring.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, API, instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
scanning, by a device, an area associated with a geographical location, the geographical location being associated with an event, the scanning comprising monitoring for detected signals from user equipment (UE) associated with a user;
detecting, by the device based on the scanning, a broadcast message from the UE;
analyzing, by the device, the broadcast message;
determining, by the device, based on the analysis, information related to vitals of the user and a location of the user within the area; and
outputting, by the device, the determined information, the output information enabling retrieval of the user to safety in response to the event.

2. The method of claim 1, wherein the broadcast message is an encrypted broadcast message communicated by the UE.

3. The method of claim 2, further comprising:
decoding the encrypted broadcast message upon detection of the encrypted broadcast message, wherein the analysis is performed in accordance with the decoded broadcast message.

4. The method of claim 1, further comprising:
detecting, over a network, the event, the detection corresponding to a time proximate to the event, wherein the scanning of the area is based on the detection of the event.

5. The method of claim 4, wherein the event is an emergency, wherein, upon occurrence of the emergency, the network is rendered inaccessible by the UE.

6. The method of claim 1, wherein the analysis of the broadcast message is based on a signal strength of the broadcast message, wherein the location of the user within the area is determined at least on the analysis of the signal strength, wherein the location of the user corresponds to a distance and direction from the device.

7. The method of claim 1, wherein the device associated with a first responder, wherein the device executes an application associated with a Cloud, wherein the user of the UE has an account with the Cloud.

8. The method of claim 1, wherein the UE is a smart ring.

9. A device comprising:
a processor configured to:
scan an area associated with a geographical location, the geographical location being associated with an event, the scanning comprising monitoring for detected signals from user equipment (UE) associated with a user;
detect, based on the scanning, a broadcast message from the UE;
analyze the broadcast message;
determine, based on the analysis, information related to vitals of the user and a location of the user within the area; and
output the determined information, the output information enabling retrieval of the user to safety in response to the event.

10. The device of claim 9, wherein the broadcast message is an encrypted broadcast message communicated by the UE.

11. The device of claim 10, wherein the processor is configured to:
decode the encrypted broadcast message upon detection of the encrypted broadcast message, wherein the analysis is performed in accordance with the decoded broadcast message.

12. The device of claim 9, wherein the processor is configured to:
detect, over a network, the event, the detection corresponding to a time proximate to the event, wherein the scanning of the area is based on the detection of the event, wherein the event is an emergency, wherein, upon occurrence of the emergency, the network is rendered inaccessible by the UE.

13. The device of claim 9, wherein the analysis of the broadcast message is based on a signal strength of the broadcast message, wherein the location of the user within the area is determined at least on the analysis of the signal strength, wherein the location of the user corresponds to a distance and direction from the device.

14. The device of claim 9, wherein the UE is a smart ring.

15. A non-transitory computer-readable storage medium tangibly encoded with computer-executable instructions that when executed by a device, perform a method comprising:
scanning, by the device, an area associated with a geographical location, the geographical location being associated with an event, the scanning comprising monitoring for detected signals from user equipment (UE) associated with a user;
detecting, by the device based on the scanning, a broadcast message from the UE;
analyzing, by the device, the broadcast message;
determining, by the device, based on the analysis, information related to vitals of the user and a location of the user within the area; and
outputting, by the device, the determined information, the output information enabling retrieval of the user to safety in response to the event.

16. The non-transitory computer-readable storage medium of claim 15, wherein the broadcast message is an encrypted broadcast message communicated by the UE.

17. The non-transitory computer-readable storage medium of claim 16, further comprising:
decoding the encrypted broadcast message upon detection of the encrypted broadcast message, wherein the analysis is performed in accordance with the decoded broadcast message.

18. The non-transitory computer-readable storage medium of claim 15, further comprising:
detecting, over a network, the event, the detection corresponding to a time proximate to the event, wherein the scanning of the area is based on the detection of the event, wherein the event is an emergency, wherein, upon occurrence of the emergency, the network is rendered inaccessible by the UE.

19. The non-transitory computer-readable storage medium of claim 15, wherein the analysis of the broadcast message is based on a signal strength of the broadcast message, wherein the location of the user within the area is determined at least on the analysis of the signal strength, wherein the location of the user corresponds to a distance and direction from the device.

20. The non-transitory computer-readable storage medium of claim 15, wherein the UE is a smart ring.
